(19) United States
(12) Patent Application Publication
(10) Pub. No.: US 2002/0165382 A1
(43) Pub. Date: Nov. 7, 2002

(54) Transcription factors that regulate normal and malignant cell growth

(76) Inventor: Craig Basson, New York, NY (US)

Correspondence Address: Daniel A. Scola, Jr., HOFFMANN & BARON, LLP, 6900 Jericho Turnpike, Syosset, NY, 11791, US

(21) Appl. No.: 10/027,059

(22) Filed: Oct. 25, 2001

Related U.S. Application Data
(60) Provisional application No. 60/243,242, filed on Oct. 25, 2000.

(51) Int. Cl. C07H 21/04; C12P 21/02; C12N 5/06; C07K 14/435

(52) U.S. Cl. 536/23.5; 530/350; 435/69.1; 435/320.1; 435/325

(57) ABSTRACT

The invention provides TBX5 protein fragments that regulate normal and malignant cell growth and nucleic acids encoding these fragments. Such protein fragments include a translated 5′ T-box sequence capable of binding to the major groove of target DNA and lack a translated 3′ T-box sequence that binds to target DNA's minor groove. In particular, the invention protein fragments that inhibit cell proliferation, this activity requiring T-box interaction with target DNA's major groove, but not target DNA's minor groove.

This invention was made with government support under NIH grant number HL03468. Accordingly, the government has certain rights in the invention.

### FIELD OF THE INVENTION

The invention relates to transcription factors. In particular, the invention relates to transcription factors that regulate cell growth.

### BACKGROUND OF THE INVENTION

Holt-Oram syndrome (or HOS) is a condition characterized by upper limb malformations and cardiac septation defects. It has been demonstrated that mutations in the human TBX5 gene underlie this disorder. The complete extent to which TBX5 functions in heart development was further established by studies in Xenopus embryos that showed that when TBX5 activity is lost, the heart fails to develop [Horb, M. and Thomsen, G. Development 126, 1739-1751 (1999)]. These studies suggest that TBX5 is necessary for normal organ development which requires regulated cell proliferation.

TBX5 was previously cloned and identified as a member of the T-box transcription factor family. Past studies have established that a highly conserved T-box domain of the TBX5 protein serves as a DNA binding domain. The highly conserved DNA binding motif (T-box or T-domain) was shown to be composed of approximately 180 amino acid residues. The T-box domain was demonstrated to bind to its target DNA as a dimer that interacts with both major and minor grooves.

Genetic data indicate that the limb and cardiac malformations that characterize Holt-Oram syndrome can be produced by nonsense mutations, deletions, insertions, rearrangements, duplications, and missense mutations in the TBX5 gene. The majority of TBX5 mutations are nonsense/frameshift mutations that introduce premature stop codons within the region of the gene that codes for the T-box domain and produce phenotypes distinct from those resulting from less common missense mutations. For example, missense mutations at the 5′ region of the T-box which disrupt T-box binding to target DNA major groove cause significant cardiac malformations, while missense mutations at the 3′ region of the T-box which disrupt T-box binding to target DNA minor groove cause extensive upper limb malformations.

While the mutations in the TBX5 gene underlying Holt-Oram syndrome have been previously characterized, little was known about the specific cellular activities regulated by TBX5 during cardiogenesis.

### SUMMARY OF THE INVENTION

In one embodiment, the invention relates to an isolated TBX5 protein fragment comprising a translated 5′ T-box sequence capable of binding to the major groove of target DNA and lacking a translated 3′ T-box sequence capable of binding to the minor groove of target DNA.

The invention further provides a cloned nucleic acid molecule encoding the TBX5 protein fragment and an expression vector capable of expressing in a host cell the TBX5 protein fragment.

In another embodiment, the invention relates to a method of inhibiting the proliferation of a cell, the method comprising introducing into the cell a polypeptide comprising a translated 5′ T-box sequence of TBX5 capable of binding to the major groove of target DNA.

Further provided by the invention is a method for identifying drug candidates that inhibit the proliferation of a cell, the method comprising measuring the effect of a compound on the proliferation of the cell, wherein compounds that inhibit the proliferation of the cell by an amount at least 10% that of a TBX5 polypeptide comprising a translated 5′ T-box sequence capable of binding to the major groove of target DNA are drug candidates.

The invention further relates to a method of stimulating growth of heart cells, the method comprising contacting the heart cells with an antagonist of a 5′ T-box sequence of the TBX5 gene or with an antagonist of the amino acids encoded by the 5′ T-box sequence a

In another embodiment, the invention relates to a method of stimulating growth of heart cells, the method comprising contacting the heart cells with an antagonist of the TBX5 gene.

A further embodiment relates to a method of identifying drug candidates that stimulate growth of heart cells, the method comprising determining whether the compounds bind to TBX5.

In another embodiment, the invention relates to a method of identifying compounds that stimulate growth of heart cells, the method comprising determining whether the compounds act, in the heart cells, as antagonists of a 5′ T-box sequence of the TBX5 gene or as antagonists of amino acids encoded by the 5′ T-box sequence.

The invention further provides a monoclonal antibody that binds specifically to an antigenic determinant in a translated 5′ T-box sequence of the TBX5 gene.

### BRIEF DESCRIPTION OF THE DRAWINGS

FIG. 1 is a schematic representation of TBX5 CDNA.

FIG. 2 is a graph showing the effect of overexpression of wild-type and mutant TBX5 isoforms on proliferation of D17 cells.

FIG. 3 is a graph showing the effect of TBX5 overexpression on cardiomyocyte proliferation in vivo.

### DETAILED DESCRIPTION OF THE INVENTION

The present invention is based on the inventor's discovery that TBX5 inhibits cellular proliferation. Preservation of the 5′ TBX5 T-box sequences, required for normal cardiogenesis and for binding to target DNA's major groove, was found to be necessary for this anti-proliferative effect. Preservation of the 3′ T-box sequences, required for normal limb morphogenesis and target DNA minor groove binding, was found not to be essential.

In particular, the invention provides protein fragments that include the translated 5′ T-box sequence required for binding to target DNA's major groove and lack the translated 3′ T-box sequence that binds to target DNA's minor groove. Further provided are cloned nucleic acid molecules encoding these fragments. The invention also encompasses methods of use of the protein fragments and nucleic acid molecules of the invention and antagonists thereof in regulating normal or malignant cell growth.

Proteins

In one embodiment, the present invention provides for an isolated TBX5 protein fragment. The fragment comprises a translated 5′ T-box sequence capable of binding to the major groove of target DNA, and lacks a translated 3′ T-box sequence capable of binding to the minor groove of target DNA.

The human TBX5 protein is the same as that reported by Basson, et al. in Nature Genetics v. 15, pp 30-35 (1997) and Proc. Natl. Acad. Sci. U.S.A., volume 96, pp 2919-2924, March 1999. The sequences reported in this paper have been deposited in the GenBank Data Base. GenBank Accession no. U80987 corresponds to the complete nucleotide coding sequence (SEQ ID NO: 2) and translated amino acid sequence (SEQ ID NO: 1) for human transcription factor TBX5.

GenBank Accession No. U89353 corresponds to an alternatively spliced form of the wild-type human transcription factor TBX5. The coding sequence and translated amino acid sequence for the alternatively spliced form are shown under SEQ ID NO: 4 and SEQ ID NO: 3, respectively.

The human wild-type TBX5 protein includes a T-box sequence that begins with amino acid 56 and ends with amino acid 238 of the human protein of SEQ ID NO: 1. A useful 5′ T-box sequence capable of binding to the major groove of target DNA begins at approximately amino acid 56 of the human protein of SEQ ID NO: 1 and contains a sufficient number of residues to inhibit cellular proliferation. Some examples of translated 5′ T-box sequences in accordance with the invention comprise approximately amino acids 56 to 100; 56 to 125; and 56 to 198 of the human TBX5 protein (SEQ ID NO: 1). The starting and ending amino acid residue of the corresponding 5′ T-box sequence in other species may correspond to positions different than those just described. However, the amino acid sequence of the T-box domain is highly conserved among species and can easily be identified by visual inspection. In addition, any number of computer programs widely available, such as SWISS-PROT and PIR, can be used to determine the sequence homology, and therefore, location of the corresponding translated 5′ T-box sequence in the other species.

The isolated TBX5 protein fragment of the present invention lacks a translated 3′ T-box sequence capable of binding to the minor groove of target DNA. In one embodiment, the protein fragment lacks the amino acid sequence including approximately amino acid 125 to the C-terminus of the human translated TBX5 or translated T-box sequence (SEQ ID NO: 1). In a further embodiment, the protein fragment lacks the sequence corresponding to amino acid 198 to the C-terminus of SEQ ID NO: 1. It is noted that the 3′ T-box domain capable of binding to the minor groove of target DNA is highly conserved among species. Therefore, the region having homology with the same region in humans can be readily determined by visual inspection or by use of standard computer programs, such as those described above

It is preferred that the TBX5 protein fragment is derived from a mammalian species. Mammals include laboratory animals, such as rats, mice, and rabbits; farm animals, such as cows, pigs, horses, and sheep; pet animals, such as dogs and cats; and primates, such as monkeys, orangutans, apes, and humans. The preferred mammals include mice or humans. The fragment may also include naturally occurring functional mutational or allelic variations.

The present invention also includes functional homologs of the protein fragments described above. The amino acid sequence of a first protein fragment is considered to be a homolog of a second amino acid sequence if the first amino acid sequence shares at least about 60% amino acid sequence identity, preferably at least about 70% amino acid sequence identity, and more preferably at least about 80% amino acid sequence identity, with the second sequence. In the case of protein fragments having high homology, the amino acid sequence of the first protein fragment shares at least about 90% amino acid sequence identity, preferably at least about 98% amino acid sequence identity, and more preferably at least about 99% amino acid sequence identity, with the amino acid sequence of the second protein fragment.

A homolog of a protein fragment is considered to be a functional homolog if the homolog maintains at least some of the activity of the protein fragment. The homolog preferably maintains at least all of the activity of the protein fragment.

In order to compare a first amino acid or nucleic acid sequence to a second amino acid or nucleic acid sequence for the purpose of determining homology, the sequences are aligned so as to maximize the number of identical and conserved character (see next paragraph) amino acid residues or nucleotides. The sequences of highly homologous proteins and nucleic acid molecules can usually be aligned by visual inspection. If visual inspection is insufficient, the nucleic acid molecules may be aligned in accordance with the methods described by George, D. G. et al., in Macromolecular Sequencing and Synthesis, Selected Methods and Applications, pages 127-149, Alan R. Liss, Inc. (1988), such as formula 4 at page 137 using a match score of 1, a mismatch score of 0, and a gap penalty of −1.

The homolog can be, for example, a substitution, addition, or deletion variant of the protein. For example, it is preferred to substitute amino acids in a sequence with equivalent amino acids. Groups of amino acids known normally to be equivalent are:

(a) Ala(A), Ser(S), Thr(T), Pro(P), Gly(G);

(b) Asn(N), Asp(D), Glu(E), Gln(Q);

(c) His(H), Arg(R), Lys(K);

(d) Met(M), Leu(L), Ile(I), Val(V); and

(e) Phe(F), Tyr(Y), Trp(W).

The TBX5 protein fragment is preferably isolated. By the term isolated it is meant that the TBX5 protein fragment is partially purified or purified to homogeneity. The protein is considered partially purified if it is at least approximately 25%, preferably at least approximately 50%, more preferably at least approximately 75%, most preferably at least approximately 90% and optimally at least approximately 95% free of other proteins.

The fragment is considered to be purified to homogeneity if it is exhibits a single band by SDS PAGE. Such fragments are preferably at least approximately 99-100% free of other proteins.

Cloned Nucleic Acid Molecules

The present invention provides a cloned nucleic acid molecule encoding any of the TBX5 protein fragments, including the functional homologs, described above. The sequence of the nucleic acid molecule may be any sequence that encodes the protein fragment. An example of such a sequence results from a naturally occurring mutation in the TBX5.2 clone, described in Basson, et al., Nature Genetics 15, pp 30-35 (1997). The mutation causes a deletion of the adenine nucleotide shown at position 593 of the complete wild-type coding sequence for human TBX5 (SEQ ID NO: 2). This mutant is referred to as ΔASN 198 FSter-TBX5 and results in truncation at amino acid 198 of the translated human protein sequence of SEQ ID NO: 1.

Preparing Protein Fragments

The protein fragment of the invention and DNA encoding the protein fragment may be chemically synthesized by methods known in the art. Suitable methods for synthesizing the protein fragment are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997). Suitable methods for synthesizing DNA are described by Caruthers in Science 230:281-285 (1985) and DNA Structure, Part A: Synthesis and Physical Analysis of DNA, Lilley, D. M. J. and Dahlberg, J. E. (Eds.), Methods Enzymol., 211, Academic Press, Inc., New York (1992).

The protein fragment may also be prepared by providing DNA that encodes the protein fragment; amplifying or cloning the DNA in a suitable host; expressing the DNA in a suitable host; and harvesting the protein. For example, the protein fragment may be translated either directly or indirectly from a cDNA encoding the TBX5 protein fragment.

The DNA encoding the protein fragment of the invention may be replicated and used to express recombinant protein following insertion into a wide variety of host cells in a wide variety of cloning vectors.

Cloning vectors may comprise segments of chromosomal, non-chromosomal, and synthetic DNA sequences. Some suitable prokaryotic cloning vectors include plasmids from E. coli, such as col E1, pCR 1, pBR 322, pMB9, pUC, pKSM, and RP4. Prokaryotic vectors also include derivatives of phage DNA such as M13 fd and other filamentous single-stranded DNA phages.

The host may be prokaryotic or eukaryotic. The DNA may be obtained from natural sources and, optionally, modified by, for example, site-specific mutagenesis. The genes may also be synthesized from the individual nucleotides in whole or in part. Synthetic methods, such as solid phase methods, are known in the art, such as those described by Caruthers in Science 230, pp 281-285 (1985) and DNA Structure Part A: Synthesis and Physical Analysis of DNA, Lilley, D. M. J. and Dahlberg, J. E. (Eds.), Methods Enzymol, 211, Academic Press, N.Y. (1992).

The invention provides for an expression vector capable of expressing in a host cell the TBX5 protein fragments described above. Vectors for expressing proteins in bacteria, especially E. coli, are also known. Such vectors include the pK 233 (or any of the tac family of plasmids), T7, pBluescript II, bacteriophage lambda, ZAP, and lambda P_{L} (Wu, R. (Ed.), Recombinant DNA Methodology II, Methods in Enzymol., Academic Press, Inc., NY, (1995). Examples of vectors with expressed fusion proteins are PATH vectors described by Dieckmann and Tzagoloff in J. Biol. Chem., vol. 260, 1513-1520 (1985). These vectors contain DNA sequences that encode anthranilate synthetase (TrpE) followed by a poly-linker at the carboxy terminus. Other expression vector systems are based on beta-galactosidase (pEX); maltose binding protein (pMAL); glutathione-S-tranferase (pGST or pGEX)—see Smith, D. B. Methods Mol. Cell Biol., vol. 4: pp 220-229 (1993); Smith, D. B. and Johnson, K. S., Gene, vol. 67, pp 31-40 (1988); and Peptide Res., vol. 3; 167 (1990), and TRX (thioredoxin) Fusion Protein (TRX FUS)—see La Vallie, R. et al., Bio/Technology, 11, pp 187-193 (1993).

Vectors useful for cloning and expression in yeast are available. Suitable examples are 2 μm circle plasmid, Ycp50, Yep24, Yrp7, Yip5, and pYAC3 [Ausubel, F. M. et al. (Eds.), Current Protocols in Molecular Biology, John Wiley & Sons, Inc., New York, (1999)].

Suitable cloning/expression vectors for use in mammalian cells are also known. Such vectors include well-known derivatives of SV-40, adenovirus, cytomegalovirus (CMV) retrovirus-derived DNA sequences. Any such vectors, when coupled with vectors derived from a combination of plasmids and phage DNA, i.e. shuttle vectors, allow for the isolation and identification of protein coding sequences in prokaryotes.

Further eukaryotic expression vectors are known in the art (e.g., P. J. Southern and P. Berg, J. Mol. Appl. Genet. 1:327-341 (1982); S. Subramani et al, Mol. Cell. Biol. 1:854-864 (1981); R. J. Kaufmann and P. A. Sharp, “Amplification And Expression Of Sequences Cotransfected with A Modular Dihydrofolate Reductase Complementary DNA Gene,” J. Mol. Biol. 159:601-621 (1982); R. J Kaufmann and P. A. Sharp, Mol. Cell. Biol. 159:601-664 (1982); S. I. Scahill et al, “Expression And Characterization Of The Product Of A Human Immune Interferon DNA Gene In Chinese Hamster Ovary Cells,” Proc. Natl. Acad. Sci. USA 80:4654-4659 (1983); G. Urlaub and L. A. Chasin, Proc. Natl. Acad. Sci. USA 77:4216-4220 (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of usefull expression control sequences are the lac system, the trp system, the tac system, the trc system, the tet system, major operator and promoter regions of phage lambda, the control region of fd coat protein, the glycolytic promoters of yeast, e.g., the promoter for 3-phosphoglycerate kinase, the promoters of yeast acid phosphatase, e.g., PhoS, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Once the gene is cloned into such an expression vector, the gene product may be produced in a suitable expression host in either a constitutive or inducible manner. Useful expression hosts include well-known prokaryotic and eukaryotic cells. Some suitable prokaryotic hosts include, for example, E. coli, such as E. coli SG-936, E. coli HB 101, E. coli W3110, E. coil X1776, E. coli X2282, E. coli DH1, E coli DH5αF′, and E. coil MRCl, Pseudomonas, Bacillus, such as Bacillus subtilis, and Streptomyces. Suitable eukaryotic cells include yeasts and other fungi, insect, animal cells, such as COS cells and CHO cells, human cells and plant cells in tissue culture.

The protein may be purified using standard known techniques. Some examples of suitable techniques include, for example, gel purification, column chromatography, or electrophoretic methods.

As described above, the expression vector according to this invention allows for the translation of a 5′ T-box domain which is capable of binding to the major groove of a target DNA and of inhibiting cellular proliferation.

Method of Inhibiting Cell Proliferation

The present invention further provides for a method of inhibiting the proliferation of a cell. In the method, a polypeptide comprising a translated 5′ T-box sequence of TBX5 capable of binding to the major groove of target DNA is introduced into the cell. The polypeptide may be the full length TBX5 protein, or a fragment, such as a fragment described above. The method may be accomplished either in vivo in a mammal or ex vivo in a cell in culture.

In the methods for inhibiting proliferation of cells described above, the cell may be any cell, such as, for example, a heart cell. The method is especially effective for inhibiting proliferation of a malignant cell. The malignant cell may be part of a solid tumor or a non-solid tumor, such as, for example, malignant bone cells. Some examples of malignant cells include, but are not limited to, carcinoma, osteocarcinoma, sarcoma, osetosarcoma, glioma, melanoma, myxoma, adenoma, or rhabdomyoma-derived cells. In particular, the cell may be a lung, breast, colon, prostate, kidney, ovary, testes, skin, heart, pancreas, thyroid, adrenal, pituitary, brain, muscle or bone cell.

In one embodiment, the method comprises introducing the TBX5 protein or protein fragment into the cell by contacting the cell with the polypeptide. Ex vivo, the cell may be contacted with the polypeptide by adding the polypeptide to a culture comprising the cells, or by adding the cells to a culture comprising the polypeptide.

In vivo, cells that are sufficiently accessible may be contacted with the polypeptide by injecting the polypeptide directly into the cell or into the extracellular space around the cell, such as into tissue or tumors comprising the cells. Some examples of cells that are sufficiently accessible include, for example, cells of the breast and skin. Some examples of cells that are less accessible, but are nevertheless still sufficiently accessible, include cells of the colon, kidney, prostate, ovaries, testes, lymph nodes, mouth, tongue, skin and throat, among others. The polypeptide may also be injected im or iv into the mammal.

In another embodiment, the method may include introducing the polypeptide into the cell by expressing in the cell a nucleic acid molecule that encodes the polypeptide. For example, the nucleic acid molecule encoding a polypeptide with a 5′ T-box domain of TBX5 capable of binding to the major groove of target DNA may be present in an expression vector that can be directly injected into accessible cells to allow for inhibition of proliferation of, for example, malignant cell types. Accessible tumors are those described above.

In one embodiment of the method described above, the translated 5′ T-box sequence is a human 5′ T-box sequence. In another embodiment, the polypeptide is a fragment that lacks a translated 3′ T-box sequence of TBX5 capable of binding to the minor groove of target DNA. Some examples of such fragments are described above.

Method of Identifying Drug Candidates That Inhibit Cell Proliferation

The invention further provides for a method of identifying drug candidates that inhibit the proliferation of cells, wherein the drug candidates are compared in performance with a standard. In this method, the standard is a TBX5 polypeptide including a translated 5′ T-box sequence capable of binding to the major groove of target DNA.

In particular, the method for identifying drug candidates that inhibit the proliferation of a cell includes measuring the effect of a compound on the proliferation of the cell and comparing it to the standard described above. In this method, the compounds that inhibit the proliferation of the cell by an amount at least 10% that of a TBX5 polypeptide including a functional 5′ T-box domain capable of binding to the major groove of target DNA, are drug candidates. Such methods of inhibiting and measuring the proliferation of a cell are well known in the art and may include in vivo or ex vivo methods.

For example, the drug candidate and TBX5 polypeptide standard may be independently incubated with a particular cell type for a sufficient period of time to inhibit the proliferation of the cells during culture. Such inhibition would be measured in relation to untreated cells. One example of a cellular proliferation assay widely used in clinical settings and laboratories is a tritiated thymidine incorporation assay.

In one embodiment of the method for identifying drug candidates that inhibit the proliferation of the cells, the compounds inhibit the proliferation of the cell by an amount at least about 25% that of the TBX5 polypeptide containing a functional 5′ T-box domain. In a further embodiment, the compounds inhibit the proliferation of the cell by an amount at least about 50% that of the TBX5 polypeptide including a functional 5′ T-box domain. The invention also provides for a method for identifying drug candidates that inhibit the proliferation of the cell wherein the compounds inhibit the proliferation by an amount at least about 75% that of the TBX5 peptide containing the functional 5′ T-box domain. In yet another embodiment, the compounds inhibit the proliferation of the cell by an amount at least about 90% that of the TBX5 polypeptide containing the functional 5′ T-box domain. In each case, a functional 5′ T-box domain is that capable of binding to the major groove of target DNA and being capable of inhibiting cellular proliferation. In a desired embodiment, the 5′ T-box domain is a human 5′ T-box domain, the sequence for which is well characterized and referenced above.

In a further embodiment of the method for identifying drug candidates, the TBX5 polypeptide, while containing a functional 5′ T-box domain, lacks a translated 3′ T-box sequence capable of binding to the minor groove of target DNA.

Method of Stimulating the Growth of Heart Cells

The present invention further provides for methods of stimulating the growth of heart cells. These methods are useful to stimulate proliferation and regeneration of normal heart muscle after heart attacks and in cardiomyopathies, for example.

One method includes contacting the heart cells with an antagonist of the TBX5 gene and, preferably, of the 5′ T-box sequence of the TBX5 gene or with an antagonist of the amino acids encoded by the 5′ T-box sequence. In one embodiment of this method, the 5′ T-box sequence encodes a protein domain of TBX5 which is capable of binding to the major groove of target DNA. The heart cells may be myocytes. Moreover, the heart cells may include fibroblasts, endothelial cells, or cardiac stem cells. In one embodiment of the method, the antagonist is a peptide antagonist that binds to native nucleic acid encoding TBX5 and thereby blocks production of TBX5 protein or affects cellular localization of TBX5 protein by interfering with TBX5 transcription, translation or transport.

The antagonist of the 5′ T-box sequence of the TBX5 gene may be an anti-sense construct. For example, anti-sense oligomers which are oligomers complementary in sequence to the cDNA sequence coding for a functional 5′ T-box domain capable of binding to the major groove of the target DNA may be used to antagonize the activity of the domain and to allow stimulation of the growth of heart cells, such as those described above. Since continued expression of TBX5 in the adult heart plays a role in the ongoing suppression of heart cell growth in the adult human, antagonists of the 5′ T-box domain responsible for major groove DNA target binding relieve this growth suppression and therefore play a role in stimulating the proliferation and regeneration of normal heart muscle after heart attacks and in cardiomyopathies, for example.

The length of the antisense oligonucleotide is not critical, as long as it is capable of hybridizing to the region of the mRNA or cDNA which encodes that part of the 5′ T-box domain of TBX5 sufficient to inhibit the proliferation of cells. The antisense oliglionucleotide should contain at least 6 nucleotides, preferably at least 10 nucleotides, and, more preferably, at least 15 nucleotides. There is no upper limit to the length of the oliglionucleotide probes. Longer probes are more difficult to prepare and require longer hybridization times. Therefore, the probe should not be longer than necessary. Normally, the oliglionucleotide probe will not contain more than 50 nucleotides, preferably not more than 40 nucleotides, and more preferably, not more than 30 nucleotides.

The antagonist used in the method for stimulating growth of heart cells provided by this invention may, as described above, be an antagonist of the protein comprising the protein fragment encoded by the 5′ T-box sequence. In one embodiment, this antagonist may be a hormone or drug inducible dominant-negative version of TBX5 protein. This dominant-negative strategy has been proven effective at inhibiting other T-domain proteins.

In one example, a damaged heart may be injected with DNA or RNA, such as mRNA, which, when translated, forms an inactive version of the TBX5 protein capable of blocking the ability of the 5′ T-box domain of endogenous TBX5 protein to bind to the major groove of target DNA. The result is stimulation of proliferation and regeneration of heart muscle, which has been the subject of a heart attack or damage due to cardiomyopathies. Hence, by blocking the ability of the endogenous TBX5 to bind to the major groove of target DNA, the effect is relieving the ongoing suppression of heart cell growth and stimulating proliferation of healthy heart cells.

In a preferred embodiment of the present invention, the antagonist is a protein that binds specifically to an epitope of the protein domain encoded by the 5′ T-box sequence of TBX5. The protein is, for example, a monoclonal antibody. In this specification, an antibody includes whole, homogenous antibodies, comprising a constant region, a framework variable region, and a hypervariable (complementarity determining) region from the same organism, such as a mouse or human. Preferably, the constant region is of human origin (e.g., a chimerized antibody); more preferably, the constant region and the framework variable region are of human origin (e.g., a humanized antibody); and most preferably, the constant region, the framework variable region, and the hypervariable region are all of human origin. Antibodies also include antibody fragments that comprise at least the hypervariable region. A preferred example of an antibody fragment is a single chain antibody. The antibodies of the invention, or derivatives of such antibodies as described above, recognize the 5′ T-box domain of the TBX5 protein with sufficient avidity to inhibit cellular proliferation.

In one embodiment, the peptide antagonist described above affects the cellular localization of TBX5 including a translated 5′ T-box sequence capable of binding to the major groove of target DNA.

For all of the aforementioned antagonists, it is necessary that they are able to inhibit binding of the TBX5 protein or protein fragment to the major groove of target DNA.

The methods of stimulating growth of heart cells provided by this invention may include stimulation of the growth ex vivo. In another embodiment, the growth may be stimulated in vivo.

In the methods of stimulating growth of heart cells provided by this invention, it is desired that the 5′ T-box sequence is a human 5′ T-box sequence. Furthermore, such methods would be most useful when contacting heart cells of a patient who has suffered a heart attack or is affected by a cardiomyopathy. The patient in each case would benefit from a stimulation of heart cell growth.

Antibodies

This invention provides antibodies, preferably monoclonal antibodies, that bind specifically to an antigenic determinant in a translated 5′ T-box sequence of the TBX5 gene. Typically, approximately six amino acids or greater form an antigenic determinant. The translated 5′ T-box sequence of the TBX5 gene is a functional 5′ T-box protein domain capable of binding to the major groove of target DNA and of inhibiting cellular proliferation.

As described above, the antibodies are preferably monoclonal, but may also be polyclonal. Monoclonal antibodies may be produced by methods known in the art. These methods include the immunological method described by Kohler and Millstein in Nature 256, pp 495-497 (1975) and by Campbell in “Monoclonal Antibody Technology”, The Production And Characterization Of Rodent And Human Hybridomas” in Burdon, et al. (Eds.), Laboratory Techniques in Biochemistry and Molecular Biology, 13, Elsevier Science Publishers, Amsterdam (1985); and Coligan, J. E., et al. (Eds.), Current Protocols In Immunology, Wiley Intersciences, NY, (1999); as well as recombinant DNA methods described by Huse, et al., Science, 246, pp 1275-1281 (1989). The recombinant DNA method preferably comprises screening phage libraries for human antibody fragments.

In order to produce monoclonal antibodies, a host mammal is inoculated with a TBX peptide or peptide fragment as described above, and then boosted. Spleens are collected from inoculated mammals a few days after the final boost. Cell suspensions from the spleens are fused with a tumor cell in accordance with the general method described by Kohler and Millstein in Nature, 256, pp 495-497 (1975).

If the fragment is too short to be immunogenic, it may be conjugated to a carrier molecular. Some suitable carrier molecules include key hold limpet, hemocyanin and bovine serum albumin. Conjugation may be carried out by methods known in the art (Coligan, J. E., et al. (Eds.), Current Protocols In Immunology, Chapter 9, Wiley Intersciences, NY, (1999). One such method is to combine a cysteine residue of the fragment with a cysteine residue on the carrier molecule.

Method of Identifying Drug Candidates That Stimulate Growth of Heart Cells

Antagonists of the 5′ T-box sequence of the TBX5 gene and antagonists of the amino acids encoded by the 5′ T-box sequence stimulate growth of heart cells, and are, therefore, potential drug candidates. The present invention provides for a method of identifying drug candidates that stimulate growth of cells wherein the method includes determining whether the compounds bind to the TBX5 protein described and referenced herein.

A binding assay may involve a single-step assay. For example, in a one-step assay, the target molecule which in this case is TBX5 protein, is immobilized and incubated with a labeled drug candidate. The labeled drug candidate binds to the immobilized TBX5 protein molecule. After washing to remove unbound molecules, the sample is assayed for the presence of the label to determine if binding has occurred and the extent to which it has occurred. Immobilization of the TBX5 protein may be accomplished in the aforementioned binding assay by immobilizing it onto a solid phase, such as a chromatography column. Such immobilization techniques are well known in the art. For example, the immobilized TBX5 may be covalently or physically bound to the solid phase support, by techniques such as covalent bonding via an amide or ester linkage or by absorption.

In the binding assay described above, the immobilized TBX5 protein and labeled drug candidate are incubated under conditions and for a period of time sufficient to allow the drug candidate to bind to the immobilized TBX5. In general, it is desirable to provide incubation conditions sufficient to bind as much of the TBX5 as possible, since this maximizes the binding of the labeled drug to the solid phase, thereby increasing the signal. The specific concentrations of the labeled drug and immobilized TBX5, the temperature and time of incubation, as well as other such assay conditions, can be varied, depending upon various factors including the concentration of the TBX5 and the sample, the nature of the sample and the like. Those skilled in the art will be able to determine operative and optimal assay conditions for each determination by employing routine experimentation.

The label may be radioactive. Some examples of useful radioactive labels include ³²p, ¹²⁵I, ¹³¹I, ³⁵S, ¹⁴C, and ³H. Use of radioactive labels have been described in U. K. 2,034,323, U.S. Pat. Nos. 4,358,535, and 4,302,204. Some examples of non-radioactive labels include enzymes and chromofores.

Some useful enzymatic labels include enzymes that cause a detectable change in a substrate. Some useful enzymes and/or substrate include for example horseradish peroxidase (pyrogallol and o-phenylenediamine), beta-galatosidase (fluorescein beta-D-galactopyranoside)-and and alkaline phosphatase (5-bromo-4-chloro-3-indolyl phosphate/nitroblue tetrazolium). The use of enzymatic labels have been described in U.K. 2,019,404, EP 63,879, in Ausubel, F. M. et al. (Eds.), Current Protocols in Molecular Biology, John Wiley & Sons, Inc., NY (1999), and by Rotman, Proc. Natl. Acad. Sci. U.S.A. 47: 1981-1991 (1961).

Useful chromophores include, for example, fluorescent, chemiluminescent and bioluminescent molecules, as well a dyes. Some specific chromophores useful in the present method of this invention include, for example, fluorescein, rhodamine, Texas red, phycoerythrin, umbelliferone, and luminol

The labels may be conjugated to the drug candidate by methods that are well known in the art. The labels may be directly attached through a functional group on the drug candidate. The drug candidate may contain or can be caused to contain a functional group. Some examples of suitable functional groups include for example, amino, carboxyl, sulfhydryls, maleimide, isocyanate, isothiocyanate.

In a preferred embodiment of the method for identifying drug candidates that stimulate the growth of heart cells, the TBX5 is a TBX5 protein fragment including a translated 5′ T-box sequence capable of binding to the major groove of target DNA and of inhibiting cellular proliferation and lacking a translated 3′ T-box sequence capable of binding to the minor groove of target DNA. This TBX5 protein fragment is described above. It is preferred that the translated 5′ T-box sequence is a human-derived sequence including approximately amino acids 56-100 of SEQ ID NO: 1. It is further preferred that the translated 3′ T-box sequence is a human-derived sequence including approximately amino acid 125 to the C-terminus of SEQ ID NO: 1 and most desirably amino acid 198 to C-terminus SEQ ID NO: 1.

A method of identifying compounds that stimulate growth of heart cells is provided herein by this invention, the method comprising determining whether the compounds act, in the heart cells, as antagonists of a 5′ T-box sequence of the TBX5 gene or as antagonists of amino acids encoded by the 5′ T-box sequence. As defined above, the 5′ T-box sequence, once translated, provides a 5′ T-box domain capable of binding to the major groove of target DNA and of inhibiting cellular proliferation. Usefull antagonists for this method are described above. One preferred antagonist would be the monoclonal antibody provided by this invention and described earlier in the specification. The actual stimulation of the growth of heart cells would be measured via a cellular proliferation assay. As described above, these methods are well known in the art and can include, for example, tritiated thymidine cellular proliferation assays. The heart cells may, for example, be myocytes or myocyte stem cells. It is well within the contemplation of the present method of this invention that the growth may be stimulated either ex vivo or in vivo.

In a desired embodiment of the method of identifying compounds that stimulate growth of heart cells, the functional 5′ T-box sequence is a human 5′ T-box sequence.

The examples which follow are set forth to aid in understanding the invention but are not intended to, and should not be construed to, limit its scope in any way. The Examples do not include detailed descriptions of conventional methods employed in the construction of vectors and plasmids, the insertion of genes encoding polypeptides into such vectors and plasmids or the introduction of plasmids into hosts.] Such methods are well known to those of ordinary skill in the art and are described in numerous publications including Sambrook, J. et al. (Eds.), Molecular Cloning: A Laboratory Manual, 2nd Edition, Cold Spring Harbor Laboratory Press, Cold Spring Harbor, N.Y. (1989) and Ausubel, F. M. et al. (Eds.), Current Protocols in Molecular Biology, John Wiley & Sons, Inc., New York (1999).

The following examples demonstrate that human TBX5 inhibits cell proliferation ex vivo, and that this activity is mediated through interactions with the T-box binding site for target DNA major groove. In addition, overexpression of recombinant human TBX5 (rhTBX5) inhibits cardiomyocyte proliferation during chick cardiogenesis. Moreover, native TBX5 expression is inversely related to cardiomyocyte proliferation during human cardiogenesis.

### EXAMPLES

### Example 1

### Preparation of TBX5 Antibody

Rabbit antiserum was developed to the TBX5 protein from human. Preimmune serum was obtained from New Zealand White rabbits which were then immunized successively with synthetic peptide NH₂-RQKVASNHSPFSSESRALC-COOH (listed under SEQ ID NO: 5 and corresponding to human TBX5 residues 264-281 plus carboxyl cysteine) [see Basson et al., Nat. Genet. 15, 30-35 (1997); and Basson et al., Proc. Natl. Acad. Sci. USA 96, 2919-2924 (1999)]. The peptide was conjugated with m-maleimidobenzoyl-N-hydroxysuccinimide ester to keyhole-limpet-hemocyanin (KLH), ovalbumin, and tetanus-toxoid (Quality Controlled Biochemicals, Hopkinton, Mass.). Rabbits were subcutaneously injected with KLH-peptide in Freund's Complete Adjuvant and boosted biweekly with immunogen in Freund's Incomplete Adjuvant. Serum titers were assessed by ELISA against peptide linked to bovine serum albumin by 4-(maleimidomethyl)cyclohexanecarboxylic-acid-N-Hydroxysuccinimide ester. High titer (>1:11,000) total rabbit antiserum was further affinity purified by incubation with peptide thiol coupled to iodylacetyl agarose, and affinity purified rabbit anti-TBX5 IgG (0.2 mg/ml) was used in further assays. 15 of 18 residues in the peptide immunogen sequence are identical in human and mouse TBX5. Affinity purified rabbit anti-human TBX5 IgG specifically recognized recombinant TBX5 (CBP-rTBX5) as determined by Western blot. Anti-TBX5 also recognized a single major polypeptide of approximately 60 kDa in murine left ventricle; the predicted murine Tbx5 Mᵣ is 58 kDa [Chapman et al., Dev. Dyn. 206, 370-390 (1996); Bruneau et al., Dev. Dyn. 211, 100-108 (1999)]. By contrast, preimmune rabbit IgG and anti-human TBX5 IgG which was preabsorbed with excess peptide antigen exhibited no reactivity against CBP-rTBX5 or native murine Tbx5. These findings demonstrate that anti-TBX5 specifically recognizes TBX5 protein.

### Example 2

### Synthesis of Recombinant TBX5

Recombinant TBX5 (58 kDa) was synthesized fused to calmodulin binding protein (CBP; 4 kDa) by subcloning TBX5.3 cDNA [Basson et al., Nat. Genet. 15, 30-35 (1997); and Basson et al., Proc. Natl. Acad. Sci. USA 96, 2919-2924 (1999)] into pCAL plasmid (Stratagene). 62 kDa CBP-rTBX5 fusion protein was induced in BL21(DE3)pLysS E. coli by IPTG and detected with biotinylated calmodulin (Stratagene).

### Example 3

### Site-Directed Mutagenesis of Human TBX5

The Gly80Arg-TBX5 mutant cDNA was generated by PCR with the QuikChange Site-Directed Mutatgenesis kit (Stratagene) per the instructions of the manufacturer. PCR-based mutagenesis of wildtype TBX5.3 cDNA in pBSIIKS-plasmid(Basson, C. T. et al., Nature Genet. 15, 30-35 (1997) was performed with two synthetic oligonucleotide primers containing the G238A transition mutation: 5′ -CATAACCAAGGCTAGAAGGCGGATGTTTC-3′ (SEQ ID NO: 6) and 5′-GAAACATCCGCCTTCTAGCCTTGGTTATG-3′ (SEQ ID NO: 7), wherein mutated bases are underlined [Basson et al., Proc. Natl. Acad. Sci. USA 96, 2919-2924 (1999)]. ΔAsn198Fster-TBX5 was a naturally occurring mutation in the TBX5.2 clone [Basson, C. T. et al., Nature Genet. 15, 30-35 (1997)]. Both mutations were confirmed by automated sequencing on an ABI 377 sequencer.

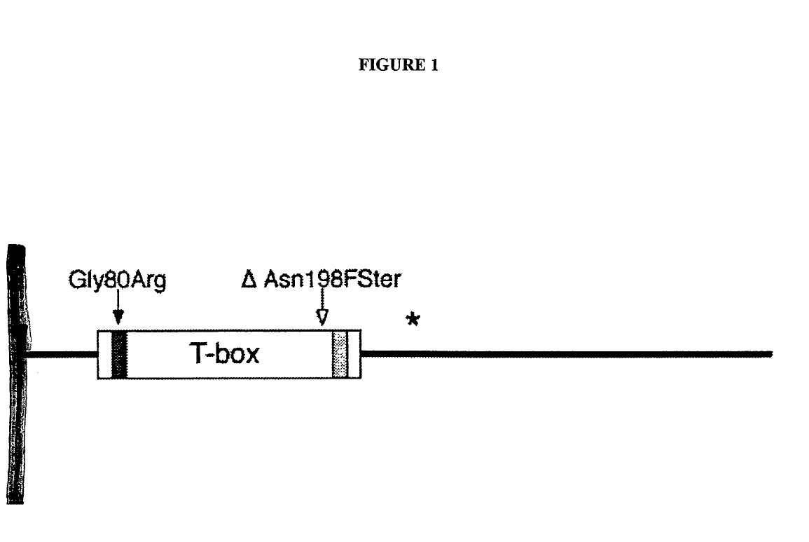

FIG. 1 is a schematic representation of TBX5 CDNA. X-ray crystallographic studies [Müller, C. and Herrmann, B., Nature, 389, 884-888 (1997)] have predicted disparate binding sites for target DNA major groove (dark gray) and for target DNA minor groove (light gray). These sites are shown relative to the positions of the Gly80Arg missense mutation (closed arrow), the ΔAsn198Fster truncation mutation (open arrow), and the anti-TBTX antibody recognition epitope (*)

### Example 4

### Retroviral TBX5 Constructs

The CXIZ retrovirus used in the present study is a replication-defective variant of the avian spleen necrosis virus. Construction of the viral vectors and propagation of the recombinant virus have been previously described [Mikawa T., Ann. N.Y. Acad. Sci. 752, 506-516 (1995) and Takebayashi-Suzuki, K. et al., Development, 127, 3523-3532 (2000)]. A 1935 bp fragment containing the entire TBX5 coding sequence (exons 2-9) was excised from wildtype or mutant TBX5.3 or TBX5.2 cDNA in pBSIIKS-plasmid with SacII and StuI. Linker adaptor pairs 5′-GGGAGCGCGCGTAATACGACTCACTATAGAACCGC-3′ (SEQ ID NO: 8)/5′-GGTTCTATAGTGAGTCGTATTACGCGCGCTCCC-3′ (SEQ ID NO: 9); 5′-GAAATCACTCCCAATTAACGCGCGAAT-3′ (SEQ ID NO: 10) /5′-CTAGATTCGCGCGTTAATTGGGAGTGATTTC-3′ (SEQ ID NO: 11) were ligated to the 5′ and 3′ ends of the TBX5 inserts, and inserts were then were subcloned into SmaI and XbaI restriction enzyme sites in the pCXIZ plasmid. TBX5 coding sequences were thereby inserted into pCXIZ between the 5′ LTR and an internal ribosome entry sequence (IRES) derived from the 5′ untranslated region of the encephalomyocarditis virus genome. Because the IRES is upstream from the bacterial lacZ gene, both the subcloned TBX5 transgene and the lacZ gene are expressed from a dicistronic mRNA. Co-translation of the transgene and β-galactosidase from the dicistronic mRNA in daughter populations from infected cells has been previously demonstrated both in tissue culture and in chick embryos. Automated sequencing on an ABI 377 sequencer of all pCXIZ plasmids confirmed the appropriate TBX5 isoform sequence. Viruses were propagated and titers assayed per published protocols [Mikawa, T., et al., Dev. Dyn. 195, 133-141 (1992); Takebayashi-Suzuki, K. et al., Development 127, 3523-3532 (2000)]. Titers of approximately 10⁶ virions/ml were obtained for all CXIZ viruses in the supernatant of each clone of virus-producing cells.

### Example 5

### Cell Proliferation

Over a 48-h period, 2×10⁵ D17 canine osteosarcoma cells or MEQC cells (a myc-transformed avian cardiomyocyte-like cell line), the gift of T. Jaffredo and A. Conrad [Jaffredo, et al; In Vitro. Exp. Cell Res 192, 481-491 (1991)], were infected with 4.5×10⁴ virions of modified CXIZ virus encoding wildtype or mutant (Gly80Arg or ΔAsn198FSter) TBX5 isoforms or unmodified CXIZ control virus in the presence of 10 μg/ml polybrene. Cells were then harvested and replated at 5×10⁴ cells per 60 mm² dish. Cells were grown in culture for one to four days, and at multiple timepoints, culture dishes were fixed with 2% paraformaldehyde and stained with X-Gal (5-bromo-4-chloro-3-indolyl-β-gal) for β-galactosidase activity. The number of β-galactosidase-positive cells/16 mm² was determined by direct visualization via light microscopy beginning 6 hours after plating.

### Example 6

### Retroviral Infection of Chick Embryos In Ovo

Media supernatants containing CXIZ viruses encoding β-galactosidase and TBX5 isoforms or, for control experiments, β-galactosidase alone were collected and concentrated by ultracentrifugation as previously described by Mikawa, T., et al., Dev. Dyn. 195, 133-141 (1992). Fertilized chick eggs obtained from an outbred flock were incubated at 37.5° C. in a humidified atmosphere until Hamburger-Hamilton (HH) Stages 17-18 [Hamburger, V. and Hamilton, H. L., J. Morph. 88, 49-92 (1951)]. A 5-10 mm diameter window was opened in the shell at the blunt end of the egg and the underlying shell membrane was removed. Ten to 100 yiral particles in 1-5 nl solution (DMEM with 7% FBS) containing 100 μg/ml polybrene were pressure-injected into the myocardium of the beating tubular heart in ovo as previously described by Mima, T., et al., Proc. Natl. Acad. Sci. 752, 506-516 (1995). Eggs were resealed with parafilm and incubated until embryos were sacrificed at E15. Six to 16 embryos were injected with each virus. In ovo infection efficiency was determined by fixing the hearts with 2% paraformaldehyde and staining overnight for β-galactosidase activity with X-Gal. CXIZ-mediated gene expression has been previously demonstrated to begin within 7-9 h of retroviral microinjections [Wei, Y. and Mikawa, T Development 127, 87-96 (2000)].

### Example 7

### Immunohistochemistry of Human and Chick Tissues

Human cardiac tissue was obtained as waste surgical pathology material from therapeutic abortions often 10-15 gestational week embryos with informed consent and approval of the Cornell Committee on Human Rights on Research. Embryonic chick cardiac tissues were obtained at E15. Tissues were paraformaldehyde-fixed and paraffin-embedded or they were flash frozen and embedded in tissue freezing medium (Fisher) for cryosectioning.

Five micron sections either were pretreated with antigen retrieval (Biogenex) and incubated with affinity purified anti-TBX5 or anti-Ki67 (Dako) or were incubated directly without pretreatment with anti-β-galactosidase (Biogenesis), or anti-PCNA (Dako). Bound primary antibody was detected with secondary antibody conjugated to horseradish peroxidase or alkaline phosphatase (LSAB2, Dako). Double immunostaining was performed with the Envision Double Staining Kit (Dako) and Vector Red alkaline phosphatase substrate (Vector).

Ten micron frozen sections were air dried and then incubated with primary antibodies: anti-atrial myosin heavy chain (AMHC1, gift of Dr. Robert Gourdie, Medical College of South Carolina; Sanders et al., Anat. Embryol. 169, 185-191 (1984), anti-actin (Sigma), or affinity-purified anti-TBX5. Bound primary antibody was detected with secondary anti-body conjugated to horseradish peroxidase (LSAB2, Dako). To detect apoptotic cells, frozen sections were fixed in paraformaldehyde and then analyzed with a modified TUNEL assay, the Dead-End™ Colormetric Apoptosis Detection System (Promega), per the instructions of the manufacturer. In brief, sections were permeabilized with proteinase K, incubated with biotinylated nucleotides and terminal deoxynucleotidyl transferase, washed in 2×SSC, quenched with 0.3% hydrogen peroxide, incubated with streptavidin-horseradish peroxidase, and product visualized with diaminobenzidine chromogen. Control sections were treated with DNase after permeabilzation.

### Example 8

### Immunohistochemistry of Cultured Cells

Cells were fixed in McClean's fixative (10 mM NaIO₄, 750 mM lysine, 37.5 mM NaPO₄, 2% paraformaldehyde) for 30 minutes on ice and then permeabilized with 0.2% Triton in PBS for 20 minutes on ice. Overnight blocking for nonspecific binding sites was performed at 4° C. in 3% bovine serum albumin/PBS. Further immunohistochemical staining was performed with affinity purified anti-TBX5 and the LSAB2 peroxidase system (Dako) per the instructions of the manufacturer. The DeadEnd™ Colorimetric Apoptosis Detection System (Promega), was used for detection of apoptotic cell death in cultured cells in the same manner as for detection in tissue sections except cells were permeabilized with Triton X-100 instead of proteinase K.

### Example 9

### RT-PCR

Total RNA was isolated from cell cultures using the RNeasy Mini Kit (Qiagen) and DNase-treated. To analyze rhTBX5 mRNA expression, 500 ng RNA was reverse transcribed and subsequently amplified by the Access RT-PCR System (Promega) with primers: TBX5ATGF (5′-ATGGCCGACGCAGACGAGGGC-3′; SEQ ID NO: 12) and TBX5AGGR (5′-AGGTCTGGTGCTGGAACATT-3′; SEQ ID NO: 13). To ensure that genomic DNA was not contaminating the total RNA sample and, thus amplified as a product of the PCR reaction, parallel control rt-PCR experiments were performed in which rt-PCR of total RNA was performed in the presence of DNA polymerase alone without reverse transcriptase. Amplified material was purified (Qiaquick, Qiagen) and a subsequent nested PCR amplification [94° C.×2 minutes followed by (94° C.×30 sec, 58° C.×30 sec, 72° C.×1 min)×35 cycles] was performed with primers: 29exF (5′-CGGGCAAAGCTGAGC-3′; SEQ ID NO: 14) and ex9R (5′-GCTGGCATACATGCAAGCTTGCCGC-3′; SEQ ID NO: 15) to amplify a 780 bp product. PCR products were analyzed on 2% agarose gels.

RT-PCR analysis of D17 and MEQC cells infected with TBX5-CXIZ viruses demonstrated the transcription of the TBX5 transgene. In particular, a 780 bp portion of TBX5 CDNA was amplified from cells infected with wild-type TBX5-CXIZ, Gly80Arg-TBX5-CXIZ, and ΔAsn198Fster-TBX5-CXIZ, but not from cells infected with the CXIZ retrovirus alone.

### Example 10

### SDS-PAGE and Western Blotting

Cultured cells were solubilized in 2% SDS. Lysate corresponding to 50 μg of total protein was electrophoresed on a 7.5% acrylamide denaturing gel and transferred to PVDF membrane. Protein expression was detected by western blotting with primary antibody: 0.2 μg/ml affinity purified anti-TBX5, 1 μg/ml anti-β-galactosidase (ICN), or 0.7 μg/ml anti-actin (Sigma). Bound primary antibody was detected by ECL (Amersham).

Western blot analysis with anti-TBX5 antibody demonstrated specific synthesis of 58 kDa rh TBX5 protein by D17 and MEQC cells infected with wild-type TBX5-CXIZ and Gly80Arg-TBX5-CXIZ retroviruses, but not by cells infected with the CXIZ retrovirus alone. It is noted that ΔAsn198FSter-TBX5-CXIZ infected cells show an absence of the 58 KDa protein and that this effect is due to the mutant's inability to be recognized by anti-TBX5 since this mutant isoform is truncated prior to the antibody recognition site (as shown in FIG. 1). Western blot analysis of infected cells for recombinant β-galactosidase showed a band corresponding to β-galactosidase (encoded by CXIZ retroviruses) for cells infected with CXIZ retroviruses. Bands of similar intensity corresponding to native actin were seen in lanes containing both uninfected and infected cells.

In Examples 11-14 below, it is demonstrated that TBX5 has a profound inhibitory effect on cell proliferation both ex vivo as well as during vertebrate embryogenesis in vivo. Mutations which modify TBX5 binding to target DNA's major groove severely compromise human cardiac development and abrogate TBX5's potency as a cellular arrest signal. The present invention provides for a TBX5 protein fragment which includes a functional 5′ T-box domain capable of binding to the major groove of target DNA and lacking a 3′ T-box domain capable of binding to the minor groove of target DNA. Such a protein fragment maintains TBX5's potency as a cellular arrest signal, as demonstrated in Examples 11-14. As shown in these examples, the specificity of TBX5's effect on cell proliferation is demonstrated by the inhibition of this effect by a missense mutation of the TBX5 major groove binding domain, but also by the observation that TBX5 mediated suppression of cell proliferation is not cell autonomous. Therefore, the inventor concludes that dysregulated cardiomyocyte proliferation contributes to cardiac malformation in Holt-Oram syndrome, and that TBX5 regulation of cellular proliferation is essential for normal cardiac growth and morphogenesis.

### Example 11

### In Vitro Analysis of TBX5 Activity

To study TBX5 effects on cell proliferation in vitro, human TBX5 isoforms were expressed in D17 canine osteosarcoma cells; canine osteosarcoma cells have previously been used to model aspects of osteoblast differentiation [Mealy et al., Cancer Lett. 126, 187-192 (1998); Shoieb et al., In Vivo, 12, 463-472 (1998)]. D17 cells were infected with CXIZ retroviruses [Mikawa et al., Dev, Dyn., 195, 133-141 (1992b); Takebayashi-Suzuki et al., Development, 127, 3523-3532 (2000)] encoding β-galactosidase and wild-type (wt) or mutant (Gly80Arg and ΔASN198FSter) TBX5 isoforms. The Gly80Arg mutation alters the 5′ end of the T-box at a putative binding domain for target DNA's major groove [Herrmann and Kispert, Trends Genet., 10, 280-286 (1994); Müller and Herrmann, Nature, 389, 884-888, (1997); Basson et al., Acad. Sci. USA, 96, 2919-2924, (1999)]. In contrast, the ΔASN198FSter mutation does not modify this domain but yields a truncated protein that lacks the 3′ end of the T-box including a putative T-box binding domain for target DNA's minor groove [Kispert and Herrmann, EMBO J., 12, 3211-3220 (1993); [Herrmann and Kispert, Trends Genet., 10, 280-286 (1994); Kispert et al., EMBO J., 14, 4763-4772 (1995); Müller and Herrmann, Nature, 389, 884-888, (1997); Li et al., Nat. Genet., 15, 21-29, (1997); Basson et al., Acad. Sci. USA, 96, 2919-2924, (1999)]. rt-PCR and Western blot analyses [Hatcher et al., Dev. Dyn., 219 90-95, (2000)] of infected D17s confirmed expression of rhTBX5, and immunohistochemistry demonstrated localization to the cell nucleus.

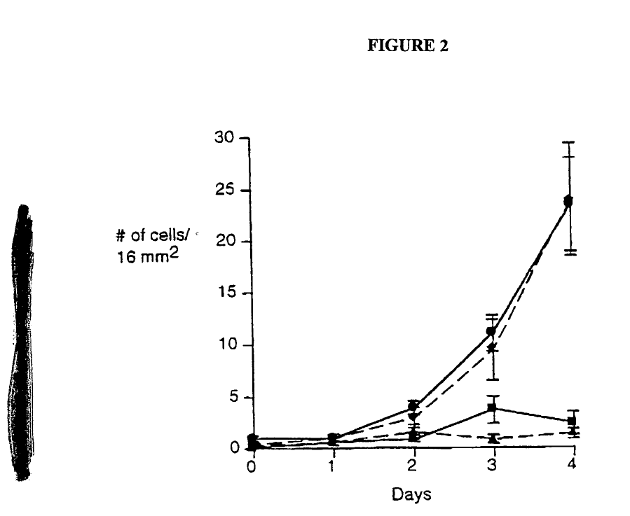

Quantification of cell proliferation (shown in FIG. 2) revealed that wt-TBX5-CXIZ-infected D17s proliferated more slowly than D17s infected with CXIZ alone. Similar inhibition of D17 proliferation results from infection with ΔASN198Fster-TBX5-CXIZ but not Gly80Arg-TBX5-CXIZ. In this experiment shown in FIG. 2, D17 cells were infected with wt-TBX5-CXIZ (solid line ▪), CXIZ alone (solid line ●), or mutant TBX5-CXIZ isoforms (dashed lines)—Gly80Arg-TBX5-CXIZ (♦) or ΔAsn198FSter-TBX5-CXIZ (▴). The number of infected D17 cells (identified by histochemical staining for β-galactosidase activity) per 16 mm² was determined by direct visualization at daily timepoints over four days. Wt-TBX5 overexpression inhibits cell proliferation as does overexpression of ΔAsn198FSter-TBX5. However, the Gly80Arg mutation blocks TBX5 mediated inhibition of D17 proliferation.

Changes in cell number were not a result of altered cell survival, since no induction of apoptosis was seen in the presence or absence of any TBX5 isoform. Thus, TBX5 inhibits cell proliferation, and this activity requires T-box interaction with target DNA's major groove but not target DNA's minor groove.

To determine if TBX5-mediated inhibition of cell proliferation was cell autonomous, the inventors assessed the proliferation rates of uninfected D17 cells cocultured with D17s infected with TBX5 retroviruses. It was observed that after 4 days of culture, uninfected D17s cocultured with wt-TBX5-CXIZ-D17s exhibited a 32% reduction (P<0.01) in cell number compared with D17s cocultured with control CXIZ-D17s. D17s cocultured with Gly80Arg-TBX5-CXIZ-D17s exhibited normal proliferation while those cocultured with ΔASN198Fster-TBX5-CXIZ-D17s exhibited a 25% reduction (P<0.01) in cell number compared with controls. Thus, the inventors concluded that TBX5 inhibition of D17 proliferation was not cell autonomous.

Because humans heterozygous for the Gly80Arg TBX5 mutation exhibit severe cardiac manifestations of Holt-Oram syndrome, and this mutation abrogates TBX5 ability to inhibit D17 cell proliferation, the inventors hypothesized that TBX5 might inhibit cardiomyocyte proliferation. As an initial in vitro test of this hypothesis, the inventors assayed the effects of TBX5 overexpression on MEQC proliferation using the same method as with D17 cells. Qualitatively similar effects were seen by TBX5 isoform overexpression on MEQCs as on D17. By 3 days of culture, MEQCs infected with wt-TBX5-CXIZ or ΔASN198Fster-TBX5-CXIZ exhibited 58 and 52% reductions in cell number, respectively, compared with CXIZ infected MEQCs (P<0.01). No significant effect on MEQC proliferation resulted from infection with Gly80Arg-TBX5-CXIZ. TBX5 inhibition of MEQC proliferation, similar to its inhibition of D17 proliferation, was not cell autonomous. Uninfected MEQCs cocultured for 3 days with wt-TBX5-CXIZ or ΔASN198Fster-TBX5-CXIZ exhibited 30 and 57% reductions in cell number, respectively, compared with those cocultured with CXIZ-infected MEQCs (P<0.01).

### Example 12

### In Vivo Analysis of TBX5 Activity During Chick Cardiogenesis

Because transformed cell lines have a limited capacity to model physiologic cardiomyocyte behavior in vivo, the inventors elected to directly explore consequences of TBX5 overexpression in vivo in embryonic chick hearts. TBX5-CXIZ was microinjected into HH stage 17-18 chick heart myocardium [Hamburger and Hamilton, J. Morph., 88, 49-92, (1951); Mikawa et al., Dev. Dyn., 195, 133-141, (1992b); Mima et al., Proc. Natl. Acad. Sci. USA, 92, 467-471 (1995); Takebayashi-Suzuki et al., Development, 127, 3523-3532, (2000)].

Whole mount staining for β-galactosidase activity at E15 confirmed mosaic expression of TBX5/β-galactosidase mRNA throughout all cardiac chambers and anti-TBX5 immunohistochemistry confirmed rhTBX5 expression by cardiomyocytes exhibiting β-galactosidase activity. Although a minority of cardiomyocytes expressed rhTBX5 isoforms, significant effects on overall cardiac morphology and size were noted. In particular, chick hearts infected with wt-TBX5 retrovirus were approximately 15% smaller than controls (see Table 1 below). Overexpression of ΔASN198Fster-TBX5, which, like wt-TBX5, inhibited proliferation ex vivo, resulted in similarly small hearts, while overexpression of Gly80Arg-TBX5, an isoform that did not alter proliferation ex vivo, had no effect on heart size. TUNEL assay demonstrated no evidence of increased apoptosis in any of the TBX5 isoform transgenic chick hearts.

TABLE 1
Effects of TBX5 Isoform Overexpression on Chick Heart Development

| | | | | ΔASN- |
|---|---|---|---|---|
| | CXIZ | | Gly80Arg- | 198FSter- |
| | Control | wt-TBX5 | TBX5 | TBX5 |
| | n = 13 | n = 16 | n = 6 | n = 12 |
| Heart weight | 70.5 ± 2.5 | 59.0 ± 1.5* | 73.5 ± 4.4 | 58.3 ± 3.0* |
| (mg) | | | | |
| Tibia length | 1.13 ± 0.04 | 1.11 ± 0.01 | 1.12 ± 0.01 | 1.13 ± 0.03 |
| mm | | | | |
| Heart weight/ | 62.3 ± 2.0 | 53.2 ± 1.3* | 64.7 ± 3.3 | 52.0 ± 2.6* |
| tibia length | | | | |
| (mg/mm) | | | | |

Data are mean ± SEM
*p < 0.01 compared with control

Microscopic analyses suggested further consequences of TBX5 overexpression. In particular, overexpression of both wt-TBX5 and ΔASN198Fster-TBX5 produced marked decreases in left and right ventricular trabeculation compared with controls, while overexpression of Gly80Arg-TBX5 did not modify trabeculation. Patchy thinning of both atrial walls was also noted in hearts infected with wt-TBX5 and ΔASN198Fster-TBX5 but not with Gly80Arg-TBX5. Just as the Gly80Arg mutation blocks TBX5 mediated inhibition of chick cardiac trabeculation, humans who are heterozygous for Gly80Arg-TBX5 exhibit abnormal isomerism with markedly increased ventricular trabeculation even in the absence of septation defects. [Basson, et al., N. Engl. J. Med. 330, 885-891 (1994); Basson, et al., Proc. Natl. Acad. Sci. USA 96, 2919-2924 (1999); Gall, et al., Am. J. Hum. Genet. 18, 187-200 (1966)].

Ventricular trabeculation during cardiogenesis has been associated with cardiomyocyte proliferation. [Sedmera et al, Anat. Rec. 258, 319-337 (2000)]. Ribozyme antagonism of the mitogen neuregulin-1 [Zhao, J. and Lemke, G. Development 125, 1899-1907 (1998)] and overexpression of the growth arrest protein Gax [Fisher, et al., Development, 124, 4405-4413 (1997)] both decrease cardiomyocyte proliferation with consequent failure of trabeculation.

### Example 13

### Effect of TBX5 Overexpression on Cardiomyocyte Proliferation In Vivo

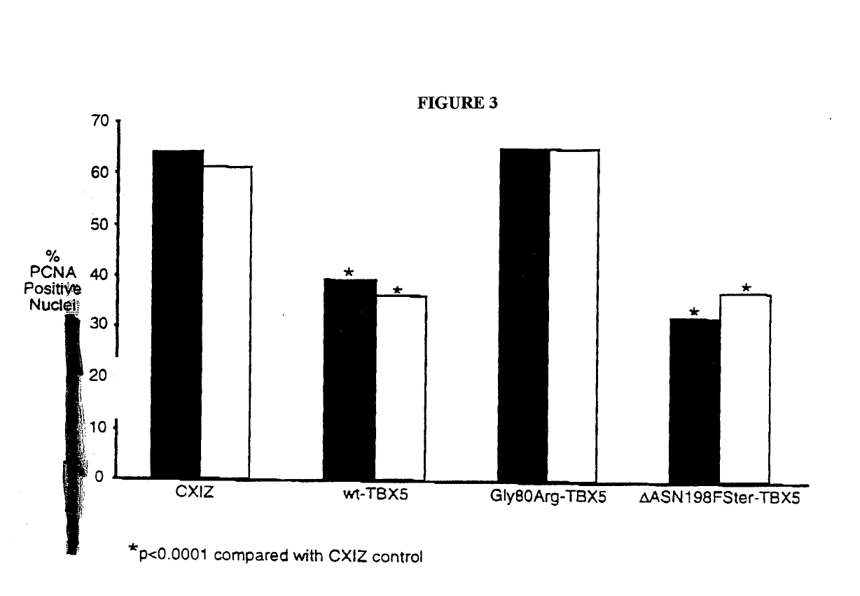

Given the inventor's observations that TBX5 inhibits MEQC proliferation in vitro and that TBX5 overexpression in vivo yields impaired heart growth with decreased ventricular trabeculation, it was hypothesized that TBX5 might alter myocardial cell proliferation in vivo. To evaluate chick cardiomyocyte proliferation in vivo, the inventor immunohistochemically assessed cardiomyocyte synthesis of proliferating cell nuclear antigen (PCNA). Embryonic chick hearts were infected with CXIZ retrovirus encoding wildtype and mutant TBX5 isoforms, animals sacrificed, and chick hearts prepared for histology. Sections of the tissue were immunostained with antibodies to beta-galactosidase and PCNA. The percentages of PCNA positive nuclei in beta-galactosidase positive cells (shown by the solid bars in FIG. 3) as well as in beta-galactosidase negative cells (shown by the open bars in FIG. 3) were calculated. For each TBX5 isoform, more than 500 infected and uninfected cells were counted from six chick hearts. p<0.0001 compared with CXIZ infected control hearts is denoted (*) in FIG. 3. The fraction of PCNA-positive cells (FIG. 3), was reduced by 40% and 50% among cardiomyocytes that overexpressed wt-TBX5 and ΔASN198Fster-TBX5 respectively compared with cardiomyocytes infected with CXIZ alone. Infection with Gly80Arg-TBX5 had no significant effect on PCNA positivity (FIG. 3). Remarkably, in all genetically manipulated chick hearts, cardiomyocytes in trabeculae without evidence of retroviral infection exhibited the same degree of PCNA positivity as those in adjacent trabeculae which did express rhTBX5 isoforms (FIG. 3). Thus, the capacity of TBX5 to inhibit cell proliferation is not cell autonomous, and the present findings suggest that TBX5 transcriptional regulation of other genes mediates paracrine signaling among cardiac cells during development.

### Example 14

### TBX5 Expression During Human Organogenesis

The ability of TBX5 to inhibit cell proliferation in vitro and during chick cardiogenesis prompted the inventor to analyze the relationship between cell proliferation and TBX5 expression during human organogenesis. TBX5 expression patterns during human embryogenesis were compared with expression patterns of two markers of cell proliferation, PCNA and Ki-67. Immunohistochemical analyses of embryonic human heart, limb, and eye revealed that regions of developing tissue which showed significant cellular proliferation, marked by either PCNA or Ki-67 staining, exhibited minimal staining for TBX5 and vice versa. TBX5 expression in the heart, as has been previously described, [Chapman, et al., Dev. Dyn. 206, 370-390 (1996); Li, et al., Nat. Genet. 15, 21-29 (1997); Bruneau, et al., Dev. Biol. 211, 100-108 (1999); Hatcher, et al., Dev. Dyn. 219, 90-95 (2000)] was highest in the atria and lowest in the ventricular myocardium, while cell proliferation was highest in the left ventricular myocardium. In the embryonic thumb, TBX5 expression was high in developing bone of the distal Phalanx and low in surrounding musculature, while cell proliferation showed the opposite pattern. Striking contrast between TBX5 expression and cell proliferation was also observed in the sensory retina of the developing eye where TBX5 was present only in the inner cell layer not in the outer cell layer to which the inventor, like others, [Gao, H. and Hollyfield, J., Dev. Biol. 169, 168-184 (1995)] have observed that cell proliferation is restricted. Thus, a reciprocal relationship was observed between TBX5 expression and cell proliferation not only in tissue culture and animal models but also during human organogenesis.

We claim:

1. An isolated TBX5 protein fragment comprising a translated 5′ T-box sequence capable of binding to the major groove of target DNA and lacking a translated 3′ T-box sequence capable of binding to the minor groove of target DNA.

2. The TBX5 protein fragment according to claim 1, wherein the protein fragment is derived from a human.

3. The TBX5 protein fragment according to claim 2, wherein the translated 5′ T-box sequence comprises approximately amino acids 56 to 100 of SEQ ID NO: 1.

4. The TBX5 protein fragment according to claim 2, wherein the protein fragment lacks the sequence comprising approximately amino acid 125 to the C-terminus of SEQ ID NO: 1.

5. The TBX5 protein fragment according to claim 2, wherein the protein fragment lacks the sequence comprising approximately amino acid 198 to the C-terminus of SEQ ID NO: 1.

6. The TBX5 protein fragment according to claim 1, wherein the translated 5′ T-box sequence comprises an amino acid sequence at least about 60% identical to amino acids 56 to 100 of SEQ ID NO: 1.

7. The TBX5 protein fragment according to claim 1, wherein the translated 5′ T-box sequence comprises an amino acid sequence at least about 80% identical to amino acids 56 to 100 of SEQ ID NO: 1.

8. The TBX5 protein fragment according to claim 1, wherein the translated 5′ T-box sequence comprises an amino acid sequence at least about 90% to about 98% identical to amino acids 56 to 100 of SEQ ID NO: 1.

9. A cloned nucleic acid molecule encoding a TBX5 protein fragment according to claim 1.

10. The nucleic acid molecule according to claim 9 wherein the protein fragment is derived from a human.

11. The nucleic acid molecule according to claim 10, wherein the translated 5′ T-box sequence comprises approximately amino acids 56 to 100 of SEQ ID NO: 1.

12. The TBX5 protein fragment according to claim 10, wherein the protein fragment lacks the sequence comprising approximately amino acid 125 to the C-terminus of SEQ ID NO: 1.

13. The nucleic acid molecule according to claim 10, wherein the protein fragment lacks the sequence comprising approximately amino acid 198 to the C-terminus of SEQ ID NO: 1.

14. The nucleic acid molecule according to claim 9, wherein the translated 5′ T-box sequence comprises an amino acid sequence at least about 60% identical to amino acids 56 to 100 of SEQ ID NO: 1.

15. The nucleic acid molecule according to claim 9, wherein the translated 5′ T-box sequence comprises an amino acid sequence at least about 80% identical to amino acids 56 to 100 of SEQ ID NO: 1.

16. The nucleic acid molecule according to claim 9, wherein the translated 5′ T-box sequence comprises an amino acid sequence at least about 90% to about 98% identical to amino acids 56 to 100 of SEQ ID NO: 1.

17. An expression vector capable of expressing in a host cell a protein fragment according to claim 1.

18. The expression vector according to claim 17, wherein the protein fragment is derived from a human.

19. The expression vector according to claim 18, wherein the translated 5′ T-box sequence comprises approximately amino acids 56 to 100 of SEQ ID NO: 1.

20. The expression vector according to claim 18, wherein the protein fragment lacks the sequence comprising approximately amino acid 125 to the C-terminus of SEQ ID NO: 1.

21. The expression vector according to claim 18, wherein the protein fragment lacks the sequence comprising approximately amino acid 198 to the C-terminus of SEQ ID NO: 1.

22. The expression vector according to claim 17, wherein the translated 5′ T-box sequence is capable of facilitating an inhibition of cellular proliferation.

23. A method of inhibiting the proliferation of a cell, the method comprising introducing into the cell a polypeptide comprising a translated 5′ T-box sequence of TBX5 capable of binding to the major groove of target DNA.

24. The method according to claim 23, wherein the polypeptide is introduced into the cell by contacting the cell with the polypeptide.

25. The method according to claim 23, wherein the polypeptide is introduced into the cell by expressing in the cell a nucleic acid molecule that encodes the polypeptide.

26. The method according to claim 23, wherein the 5′ T-box sequence is a human 5′ T-box sequence.

27. The method according to claim 23, wherein the polypeptide lacks a translated 3′ T-box sequence of TBX5 capable of binding to the minor groove of target DNA.

28. The method according to claim 23, wherein the cell is a malignant cell.

29. The method according to claim 28, wherein the malignant cell is a carcinoma, osteocarcinoma, sarcoma, osteosarcoma, glioma, melanoma, myxoma, adenoma, or rhabdomyoma-derived cell.

30. The method according to claim 23, wherein the cell is a lung, breast, colon, prostate, kidney, ovary, testes, skin, heart, pancreas, thyroid, adrenal, pituitary, brain, muscle or bone cell.

31. The method according to claim 23, wherein the cell is a metastasized cell.

32. The method according to claim 23, wherein the proliferation is inhibited ex vivo.

33. The method according to claim 23, wherein the proliferation is inhibited in vivo.

34. The method according to claim 23, wherein the cell is a post-embryonic cell.

35. A method for identifying drug candidates that inhibit the proliferation of a cell, the method comprising measuring the effect of a compound on the proliferation of the cell, wherein compounds that inhibit the proliferation of the cell by an amount at least 10% that of a TBX5 polypeptide comprising a translated 5′ T-box sequence capable of binding to the major groove of target DNA are drug candidates.

36. The method according to claim 35, wherein the compounds inhibit the proliferation of the cell by an amount at least about 25% that of the TBX5 polypeptide.

37. The method according to claim 35, wherein the compounds inhibit the proliferation of the cell by an amount at least about 50% that of the TBX5 polypeptide.

38. The method according to claim 35, wherein the compounds inhibit the proliferation of the cell by an amount at least about 75% that of the TBX5 polypeptide.

39. The method according to claim 35, wherein the compounds inhibit the proliferation of the cell by an amount at least about 90% that of the TBX5 polypeptide.

40. The method according to claim 35, wherein the polypeptide lacks a translated 3′ T-box sequence capable of binding to the minor groove of target DNA.

41. The method according to claim 35, wherein the proliferation is inhibited ex vivo.

42. The method according to claim 35, wherein the proliferation is inhibited in vivo.

43. The method according to claim 35, wherein the 5′ T-box sequence is a human 5′ T-box sequence.

44. A method of stimulating growth of heart cells, the method comprising contacting the heart cells with an antagonist of a 5′ T-box sequence of the TBX5 gene or with an antagonist of the amino acids encoded by the 5′ T-box sequence.

45. The method according to claim 44, wherein the 5′ T-box sequence encodes a protein domain of TBX5 capable of binding to the major groove of target DNA.

46. The method according to claim 44, wherein the cells are myocytes.

47. The method according to claim 44, wherein the cells are fibroblasts, endothelial cells, or cardiac stem cells.

48. The method according to claim 44, wherein the antagonist of the 5′ T-box sequence of the TBX5 gene is an anti-sense construct.

49. The method according to claim 44, wherein the antagonist of amino acids encoded by the 5′ T-box sequence is a hormone-inducible or drug-inducible dominant negative version of TBX5 protein.

50. The method according to claim 44, wherein the antagonist of amino acids encoded by the 5′ T-box sequence is a monoclonal antibody.

51. The method according to claim 44, wherein the growth is stimulated ex vivo.

52. The method according to claim 44, wherein the growth is stimulated in vivo.

53. The method according to claim 44, wherein the 5′ T-box sequence is a human 5′ T-box sequence.

54. The method according to claim 44, wherein the heart cells are in a patient who has suffered a heart attack or is affected by a cardiomyopathy.

55. A method of stimulating growth of heart cells, the method comprising contacting the heart cells with an antagonist of the TBX5 gene.

56. The method according to claim 55, wherein the antagonist of the TBX5 gene is a peptide antagonist.

57. The method according to claim 56, wherein the peptide antagonist affects cellular localization of TBX5 including a translated 5′ T-box sequence capable of binding to the major groove of target DNA.

58. A method of identifying drug candidates that stimulate growth of heart cells, the method comprising determining whether the compounds bind to TBX5.

59. The method according to claim 58, wherein the TBX5 comprises a translated 5′ T-box sequence capable of binding to the major groove of target DNA and lacks a translated 3′ T-box sequence capable of binding to the minor groove of target DNA.

60. The method according to claim 59, wherein the translated 5′ T-box sequence is a human sequence comprising approximately amino acids 56 to 100 of SEQ ID NO: 1.

61. The method according to claim 60, wherein the TBX5 lacks the sequence comprising approximately amino acid 125 to the C-terminus of SEQ ID NO: 1.

62. The method according to claim 60, wherein the TBX5 lacks the sequence comprising approximately amino acid 198 to the C-terminus of SEQ iID NO: 1.

63. A method of identifying compounds that stimulate growth of heart cells, the method comprising determining whether the compounds act, in the heart cells, as antagonists of a 5′ T-box sequence of the TBX5 gene or as antagonists of amino acids encoded by the 5′ T-box sequence.

64. The method according to claim 63, wherein the heart cells are myocytes or myocyte stem cells.

65. The method according to claim 63, wherein the growth is stimulated ex vivo.

66. The method according to claim 63, wherein the growth is stimulated in vivo.

67. The method according to claim 63, wherein the 5′ T-box sequence is a human 5′ T-box sequence.

68. A monoclonal antibody that binds specifically to an antigenic determinant in a translated 5′ T-box sequence of the TBX5 gene.

69. The monoclonal antibody according to claim 68, wherein the 5′ T-box sequence encodes a protein domain capable of major groove target DNA binding.


SEQUENCE LISTING (15 sequences)

SEQ ID NO: 1 - protein, 349 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKGECDHPWSICFLSYLFLSLGWG

SEQ ID NO: 2 - DNA, 1050 bp, Homo sapiens
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaaggtgagtgtgatcacccctggtcaatttgctttctttcttaccttttcctttccttgggttgggggtga

SEQ ID NO: 3 - protein, 518 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 4 - DNA, 1557 bp, Homo sapiens
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaa

SEQ ID NO: 5 - protein, 19 aa, Artificial Sequence
  synthetic peptide
RQKVASNHSPFSSESRALC

SEQ ID NO: 6 - DNA, 29 bp, Artificial Sequence
  synthesized primer
cataaccaaggctagaaggcggatgtttc

SEQ ID NO: 7 - DNA, 29 bp, Artificial Sequence
  synthetic sequence
gaaacatccgccttctagccttggttatg

SEQ ID NO: 8 - DNA, 35 bp, Artificial Sequence
  synthetic sequence
gggagcgcgcgtaatacgactcactatagaaccgc

SEQ ID NO: 9 - DNA, 33 bp, Artificial Sequence
  synthetic sequence
ggttctatagtgagtcgtattacgcgcgctccc

SEQ ID NO: 10 - DNA, 27 bp, Artificial Sequence
  synthetic sequence
gaaatcactcccaattaacgcgcgaat

SEQ ID NO: 11 - DNA, 31 bp, Artificial Sequence
  synthetic sequence
ctagattcgcgcgttaattgggagtgatttc

SEQ ID NO: 12 - DNA, 21 bp, Artificial Sequence
  synthetic primer
atggccgacgcagacgagggc

SEQ ID NO: 13 - DNA, 20 bp, Artificial Sequence
  synthetic sequence
aggtctggtgctggaacatt

SEQ ID NO: 14 - DNA, 15 bp, Artificial Sequence
  synthetic sequence
cgggcaaagctgagc

SEQ ID NO: 15 - DNA, 25 bp, Artificial Sequence
  synthetic sequence
gctggcatacatgcaagcttgccgc